(19) United States
(12) Patent Application Publication
(10) Pub. No.: US 2007/0054859 A1
(43) Pub. Date: Mar. 8, 2007

(54) Estrogen receptor-related receptor alpha (ERRalpha) and cartilage formation

(76) Inventors: Jane E. Aubin, Toronto (CA); Edith Bonnelye, Lyon (FR)

Correspondence Address: MYERS BIGEL SIBLEY & SAJOVEC, PO BOX 37428, RALEIGH, NC, 27627, US

(21) Appl. No.: 11/588,650

(22) Filed: Oct. 27, 2006

Related U.S. Application Data
(62) Division of application No. 10/116,304, filed on Apr. 4, 2002, now abandoned.
(60) Provisional application No. 60/281,023, filed on Apr. 4, 2001.

(51) Int. Cl. A61K 38/22; A61K 31/56

(52) U.S. Cl. 514/12; 514/182

(57) ABSTRACT

Estrogen related receptor α (ERRα) is involved in control of cartilage formation in mammals. Increasing ERRα activity causes stimulation of cartilage formation, providing a means of therapeutic intervention in diseases such as arthritis which involve cartilage destruction. Compounds may be screened for their potential as therapeutics by screening their effect on ERRα cartilage promoting activity.

### RELATED APPLICATION INFORMATION

This application is a divisional of U.S. application Ser. No. 10/116,304, filed Apr. 4, 2002 (currently pending), which application claims the benefit of U.S. Provisional Application Ser. No. 60/281,023 filed Apr. 4, 2001, the disclosures of which are incorporated by reference herein in their entireties.

### FIELD OF THE INVENTION

The present invention relates to methods and pharmaceutical preparations for modulation of cartilage formation.

### BACKGROUND OF THE INVENTION

In the description which follows, references are made to certain literature citations which are listed at the end of the specification and all of which are incorporated herein by reference.

Nuclear receptors are transcription factors involved in various physiological regulatory processes. The superfamily to which nuclear receptors belong comprises both ligand-dependent molecules such as the steroid hormone-, thyroid hormone-, retinoic acid- and vitamin D-receptors, and an increasing number of so-called orphan receptors for which no ligand has yet been determined [Gronemeyer, 1995; Enmark, 1996]. Indeed, it is not yet known whether the orphan receptors have ligands that await identification or whether they act in a constitutive manner. The orphan receptors display the same structural organization as do the classic ligand-dependent receptors: the A/B domain located in the N-terminal part of the protein harbors a ligand-independent transactivation function (AF-1); the C domain, which is the most conserved part of the molecule, is responsible for the specific DNA-binding activity; the E domain contains the ligand binding hydrophobic pocket and contributes to receptor dimerization and to the ligand-dependent transactivation function (AF-2).

Two orphan receptors, estrogen receptor-related receptor α (ERRα) and ERRβ ([Giguere, 1988]; NR3B1 and NR3B2, respectively, according to the Nuclear Receptors Nomenclature Committee, 1999) are closely related to the estrogen receptors ERα and ERβ [Green, 1986; Kuiper, 1996]; NR3A1 and NR3A2 respectively). ERRα and ERRβ were identified by low-stringency screening of cDNA libraries with a probe encompassing the DNA-binding domain of the human estrogen receptor (ER). Recently, a third estrogen receptor-related receptor, ERR3 or ERRβ was identified by yeast two-hybrid screening with the giucocorticoid receptor interacting protein 1 (GRIP1) as bait [Hong, 1999]. The DNA binding domain region of ERRs and ERs is highly conserved, however the others parts of the protein share very little homology [Giguere, 1988; Hong, 1999]. Therefore, sequence alignment of ERRα and the ERs reveals a high similarity (68%) in the 66 amino acids of the DNA-binding domain and a moderate similarity (36%) in the ligand-binding E domain, which may explain the fact that ERRα does not bind estrogen. Although ligands for the ERRs have not been clearly identified, the pesticides chlordane and toxaphene have been reported to be antagonists of ERRα [Yang, 1999]. Yang et al. also showed that ERRα modulates the activating effect of estrogens lactoferrin promoter and suggested that ERRα may interact with ERs through protein-protein interaction [Yang, 1996; Zhang, 2000].

ERRα has also been described as a modulator of the human aromatase gene in breast, and hypothesized to be critical for normal breast development and to play an important role in the pathogenesis and maintenance of breast cancer via its ability to interact with ERs [Yang, 1998]. Aromatase cytochrome p450 catalyzes the conversion of androgens (C19 steroids) to estrone, the immediate precursor of estradiol. Aromatase cytochrome p450 is the product of the CYP19 gene which exhibits tissue specific expression through the use of different promoters [Simpson, 1997; Simpson, 2000]. The CYP19 gene has been linked to rheumatoid arthritis susceptibility [John, 1999]. In aged orchidectomized rats, administration of the aromatase inhibitor vorozole increased bone resorption and increased bone loss suggesting that aromatase activity (i.e., the ability to convert androgens to estrogens) is required through life to maintain proper bone homeostasis [Vanderschueren, 1996; Vanderschueren, 2000]. Skeletal defects associated with deficiency of aromatase in humans are noted at puberty and are associated with continued longitudinal growth (i.e. failure to close growth plate) amongst other problems. This is consistent with the observation that aromatase is present in articular chondrocytes [Sasano, 1997] suggesting a dependence on aromatase activity for proper cartilage development and homeostasis.

Due to their homology to the ERs, it is possible that the ERRs may intervene in the signals induced by estrogen in cartilage. ERRβ expression, however, is restricted to early development and to a few adult tissues [Giguere, 1988; Pettersson, 1996]. In contrast, ERRα has a broader spectrum of expression, including fat, muscle, brain, testis and skin [Bonnelye, 1997]. Strikingly, ERRα is also highly expressed in the ossification zones of the mouse embryo (in long bones, vertebrae, ribs and skull), and is more widely distributed in osteoblast-like cells than is ERα [Bonnelye, 1997]. Moreover it has been shown that ERRα positively regulates the osteopontin gene [Vanacker, 1998], an extracellular matrix molecule secreted by osteoblasts and thought to play a role in bone remodeling [Denhardt, 1998].

It has been shown that upregulation of ERRα increased osteoblast differentiation from progenitor cells and proliferation of progenitor cells in mammals, while down regulation of ERRα caused inhibition of bone formation, with reduction of osteoblast numbers and differentiation. (International Patent Application No. PCT/CA00/01015).

ERRα was shown to be expressed also in osteocytes in both calvaria and long bones, indicating a role in skeletal maintenance.

It is clear from human and animal studies that destruction of cartilage occurs in rheumatoid arthritis and other inflammatory arthrides. Available treatments are generally based on administration of anti-inflammatory agents to reduce symptoms and no therapies are available which act at the level of cartilage, to promote restoration of the damaged tissue.

No involvement of ERRα in cartilage formation and maintenance has been previously described.

### SUMMARY OF THE INVENTION

The present inventors have shown that ERRA is highly expressed during chondrogenesis and plays a physiological role in cartilage formation at both proliferation and differentiation stages. ERRα has been shown to have an important function in the formation and turnover of cartilage, including articular surfaces.

Stimulating ERRα expression or activity promotes cartilage formation and antagonising ERRα expression or activity inhibits cartilage formation.

These findings enable therapeutic intervention to promote cartilage formation where this is desirable, for example in conditions involving cartilage loss or destruction, by increasing ERRα cartilage promoting activity.

Interventions to inhibit cartilage formation, for example in chondrosarcomas or chondrodysplasias, are also enabled, by reducing ERRα cartilage promoting activity.

One embodiment of the invention is use of an agent selected from the group consisting of:
(a) an estrogen receptor-related receptor alpha (ERRα) agonist;
(b) a substantially purified ERRα protein; and
(c) a nucleotide sequence encoding ERRα protein or an effective portion thereof; and
(d) an agent which enhances expression of a gene encoding an ERRα protein for the preparation of a medicament for promoting cartilage formation in a mammal.

A further embodiment is a method for promoting cartilage formation in a tissue or cell in vitro comprising contacting the tissue or cell with an agent selected from the group consisting of:
(a) an ERRα agonist;
(b) a substantially purified ERRα protein;
(c) a nucleotide sequence encoding ERRα protein or an effective portion thereof; and
(d) an agent which enhances expression of a gene encoding an ERRα protein.

A further embodiment is use of an agent selected from the group consisting of:
(a) an ERRα antagonist;
(b) a purified antibody which binds specifically to ERRα protein;
(c) an antisense nucleotide sequence complementary to and capable of hybridizing to a nucleotide sequence encoding ERRα protein; and
(d) an agent which reduces expression of the gene encoding ERRα protein
   for the preparation of a medicament for inhibiting cartilage formation in a mammal.

A further embodiment is a method of promoting cartilage formation in a mammal comprising administering to the mammal an effective amount of an agent selected from the group consisting of:
(a) an estrogen receptor related receptor alpha (ERRα) agonist;
(b) a substantially purified ERRα protein
(c) a nucleotide sequence encoding ERRα protein; and
(d) an agent which enhances expression of a gene encoding an ERRα protein.

A further embodiment is a method of inhibiting cartilage formation in a mammal comprising administering to the mammal an effective amount of an agent selected from the group consisting of:
(a) an ERRα antagonist;
(b) a purified antibody which binds specifically to an ERRα protein;
(c) an antisense nucleotide sequence complementary to and capable of hybridizing to a nucleotide sequence encoding an ERRα protein; and
(d) an agent which reduces expression of a gene encoding an ERRα protein.

A further embodiment is a method for screening a candidate compound for its ability to modulate ERRα cartilage promoting activity comprising:
(a) providing an assay system for measuring cartilage formation;

and
(b) measuring the cartilage promoting activity of ERRα in the presence or absence of the candidate compound,
   wherein a change in ERRα cartilage promoting activity in the presence of the compound relative to ERRα cartilage promoting activity in the absence of the compound indicates an ability to modulate ERRα cartilage promoting activity.

Compounds which effect modulation of the cartilage promoting activity of ERRα may be useful to promote cartilage formation, if their effect is positive, or to inhibit cartilage formation, if their effect is negative.

In accordance with another embodiment of the present invention, a pharmaceutical composition comprises a chondrogenesis promoting amount of an agent selected from the group consisting of:
(a) an ERRα agonist;
(b) a substantially purified ERRα protein;
(c) a nucleotide sequence encoding ERRα protein or an effective portion thereof; and
(d) an agent which enhances expression of a gene encoding an ERRα protein; and
   a pharmaceutically acceptable carrier.

In accordance with another embodiment of the present invention, a pharmaceutical composition comprises a cartilage formation inhibiting amount of an agent selected from the group consisting of:
(a) an ERRα antagonist;
(b) a purified antibody which binds specifically to ERRα protein;
(c) an antisense nucleotide sequence complementary to and capable of hybridizing to a nucleotide sequence encoding ERRα protein; and
(d) an agent which reduces expression of the gene encoding ERRα protein
   and a pharmaceutically acceptable carrier.

### SUMMARY OF THE DRAWINGS

Certain embodiments of the invention are described, reference being made to the accompanying drawings, wherein:

FIG. 1, Panel A is a Northern blot showing expression, in C5.18 cells, of ERRα, link protein, L32 and aggrecan over a proliferation-differentiation time course in presence (+Dex) or absence (−Dex) of dexamethasone (Dex) during proliferation (day 5), early cartilage nodule formation (days 9, 11) and late (day 17) cartilage nodule formation.

FIG. 1, Panel B shows ERRα mRNA expression normalized against that of the ribosomal protein L32; the Y-axis is the ratio of the ERRα signal to that of L32. For comparison, mRNA levels for two chondroblast markers, aggrecan and link protein, are also shown (Panel A) and normalized against L32 (Panel B).

FIG. 2, Panel A, shows ERRα expression in C5.18 cells cultured with dexamethasone, determined as mRNA level by semi-quantitative RT-PCR and normalised against expression of the ribosomal probe L32 (Y axis-Markers/L32), at days 3, 6, 11, 15 and 21 of culture. Panels B, C and D show the expression of three chondroblast markers, aggrecan, type 11 collagen and link protein, respectively, also normalised against L32.

FIG. 3 shows proliferation of C5.18 cells treated with antisense (AS) or sense (S) oligonucleotides at 1 μM, 2 μM or 5 μM or with no (Ct) oligonucleotide during the proliferation stage (days 1-4). Data are expressed as mean cell number +/−SD of triplicate wells.

FIG. 4 shows cartilage nodule formation in cultures of C5.18 cells treated with antisense (AS) or (S) oligonucleotides at 0.5 μM, 1 μM and 2 μM or no (Ct) oligonucleotide during the differentiation time period (days 6-11).

FIG. 5 shows cartilage formation (expressed as number of cartilage nodules/dish) in C5.18 cells transfected with pcDNA3-ERRα (ERRα vector) or pcDNA3 empty plasmid (empty vector).

FIG. 6, Panel A, shows ERRα expression, determined as mRNA levels by semi-quantitative RT-PCR and normalised against expression of the ribosomal probe L32, in two joints from each of three control rats, C1, C2 and C3, and three rats with arthritis (A1, A2 and A3). Panel B shows ERRα expression, similarly determined, in data pooled from the six control joints (controls) and the six arthritic joints (arthritis).

FIG. 7 shows ERRα expression, determined and expressed as for FIG. 6, in a femoral bone from each of three control mice (1, 2, 3) and three arthritic mice (4, 5, 6) and in pooled joints from three control mice (7) and three arthritic mice (8).

FIG. 8 shows ERRα expression, determined and expressed as for FIG. 6, in C5.18 cell cultures grown—Panel A: in the presence (+) or absence (−) of fetal bovine serum (FBS) and Panel B: in the presence of estrogen (10⁻⁹M E2) or 0.01% ethanol vehicle (VEH). * p<0.01; ** p<0.005; ns p<0.06.

### DETAILED DESCRIPTION OF THE DRAWINGS

The present inventors have found a new role for the orphan receptor, estrogen receptor-related receptor α (ERRα), in the modulation of cartilage growth and differentiation in mammals.

Cartilage formation involves the proliferation of chondroprogenitor cells and their differentiation first into chondroblasts and then into mature chondrocytes which synthesise and deposit cartilage.

Studies of a fetal rat chondrogenic cell line, which is an accepted model of mammalian chondroprogenitor proliferation and differentiation into chondrocytes, with formation of cartilage nodules, (Grigoriadis 1996; McDougall 1996) showed that ERRα was expressed throughout the process of cartilage formation, from early chondroprogenitor cells in the perichondrium to mature cartilage-synthesising chondrocytes.

Stimulation of ERRα expression and increased ERRα activity gave both increased chondroprogenitor cell and chondrocyte proliferation and increased differentiation of chondroprogenitors into mature chondrocytes.

Inhibition of ERRα expression and reduced ERRα activity gave decreased proliferation of chondroprogenitors and chondrocytes and decreased differentiation and cartilage nodule formation.

Similar results were found in vivo, in both fetal and adult rat cartilage, where ERRα expression was high in both progenitor cells and cartilage-synthesising cells in the cartilage of tibia and metatarsal bones. The presence of the ERRα receptor in both articular and growth plate chondrocytes suggests a role for ERRα both in cartilage formation and its maintenance and integrity throughout the lifetime of the mammal. This role is further supported by the inventors'findings that ERRα expression was decreased in the eroding articular cartilage of rats and mice suffering from induced arthritis, in several accepted models of human inflammatory arthritis.

The invention provides methods and pharmaceutical compositions for promoting cartilage formation in a mammal by increasing ERRα activity. As used herein, “ERRα activity” means ERRα chondrogenic or cartilage promoting activity, ie. stimulation of cartilage production, which may occur by stimulation of proliferation of chondroprogenitor cells and/or chondrocytes and/or promotion of differentiation of chondroprogenitor cells and/or stimulation of chondrocytes to increase cartilage formation.

ERRα activity may be increased in a mammal by increasing the amount of ERRα protein present or by increasing the chondrogenic effect of existing ERRα protein. Increased ERRα activity may be achieved, for example, by up regulating expression of the ERRα gene, by gene therapy to provide a nucleotide sequence encoding ERRα protein, by administering an agent which enhances ERRα expression, by administering ERRα protein or by administering an ERRα agonist. An ERRα agonist is a compound which increases the chondrogenic activity of ERRα protein.

Agents which increase ERRα activity may be used for preparation of medicaments for promoting cartilage formation.

One compound which has been shown by the inventors to increase ERRα expression is estrogen. Estrogen analogues, including selective estrogen receptor modifiers (SERMS), may be screened by the methods described herein to select those active as ERRα agonists or ERRα expression up-regulators.

The cartilage formation promoting methods and compositions of the invention can be employed to treat conditions associated with cartilage loss, cartilage degeneration or cartilage injury. Such conditions include the various disorders described collectively as arthritis.

Arthritis is a term used to designate generally diseases of the joint . Arthritis includes many different conditions but is characterized generally by the presence of joint inflammation. Inflammation is involved in many forms of arthritis and results, among other things, in the destruction of joint cartilage.

The list of diseases that are included in the term arthritis includes, but is not limited to, ankylosing spondylitis, childhood arthritis, chronic back injury, gout, infectious arthritis, osteoarthritis, osteoporosis, pagets's disease, polymyalgia rheumatica, pseudogout, psoriatic arthritis, reactive arthritis, reiter's syndrome, repetitive stress injury, and rheumatoid arthritis.

Cartilage destruction or injury can also result from joint surgery, joint injury and obesity.

A number of symptomatic treatments for arthritis exist, including analgesics and non-steroidal anti-inflammatory agents. Other treatments for inflammatory arthritis include disease modifying agents (DMARDS) such as gold salts, methotrexate, sulfasalazine, hydroxychloroquine, chloroquine and azathioprine. Steroids and corticosteroids are anti-inflammatory agents that are used to treat the inflammation underlying cartilage destruction.

No current arthritis therapy acts at the level of cartilage. Although many of the treatments for arthritis may be able to reduce the effects of the inflammation which causes cartilage destruction, these treatments do not promote cartilage regrowth in the affected tissue.

The present invention provides methods and pharmaceutical compositions for treating arthritis by increasing ERRα activity. ERRα activity may be increased as described above.

An ERRα agonist or an agent which enhances ERRα expression, such as estrogen, may be administered systemically to the subject in need of treatment, or may be administered locally, for example by intra-articular injection.

If ERRα activity is to be increased by gene therapy, a preferred method is by administration of a suitable vector, such as an adenovirus or an adeno-associated virus carrying the ERRα gene, by intra-articular injection. Such intra-articular gene administration has been described by Goater et al., (2000) and van Lent et al. (2002).

A further preferred method is the ex vivo transfection of mesenchymal stem cells or chondroprogenitor cells with the ERRα gene, followed by intra-articular injection of the treated cells. Such techniques have been described by Nixon et al., (2000) and Gelse et al., (2001).

ERRα protein was found widely distributed in vitro in C5.18 cell cultures from early proliferation stages through cartilage nodule formation. ERα and ERβ were also detected in C5.18 cells at all times analysed, although ERβ was present at somewhat lower levels and in a more patchy appearance. These results indicate that ERRα and ERα and/or ERβ are co-expressed in chondrogenic cells, and that these receptors may act alone or together to regulate the expression of target genes in cartilage.

The role played by expression of the estrogen receptors in chondrocytes has been unclear. The data indicate that ERRα and one or both of the ERs are co-expressed chondrogenic cells. Protein analysis provided the result that ERRα and ERα are co-distributed in large cohorts of chondrogenic cells, suggesting that these receptors may regulate the expression of the same target genes in cartilage. This may occur via their known ability to participate in protein-protein interactions and their recently described capacity to bind to the same DNA target (SFRE and ERE) sequence on the osteopontin promoter. ERRα and ERβ co-expression also occurs in some chondrogenic cells, but interactions between these two receptors has not yet been described, although they have recently been described to recognize the same ERE response element. These data suggest that ERRα , ERα and ERβ are co-expressed in chondrogenic cells, and may display at least some functions in common, either singly or through their interactions, with regulatory capacities to act on target genes.

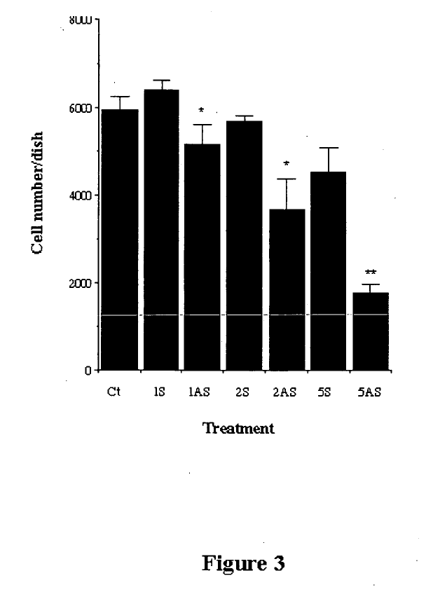

Consistent with its expression in proliferating chondrogenic C5.18, it was found that antisense oligonucleotide-induced downregulation of ERRα inhibited proliferation of C5.18 cell populations as illustrated in FIG. 3. This decrease in proliferation was an unexpected result, given the previous observation that ERRα expression appeared to correlate with exit from proliferation and the onset of the differentiation process in at least certain other cell types, including the nervous system, the epidermis and muscles in the developing mouse [Bonnelye, 1997]. This surprising result suggests that ERRα may play cell-type specific functions. Cell-type specific treatments can thus be developed for particular cartilage/joint diseases.

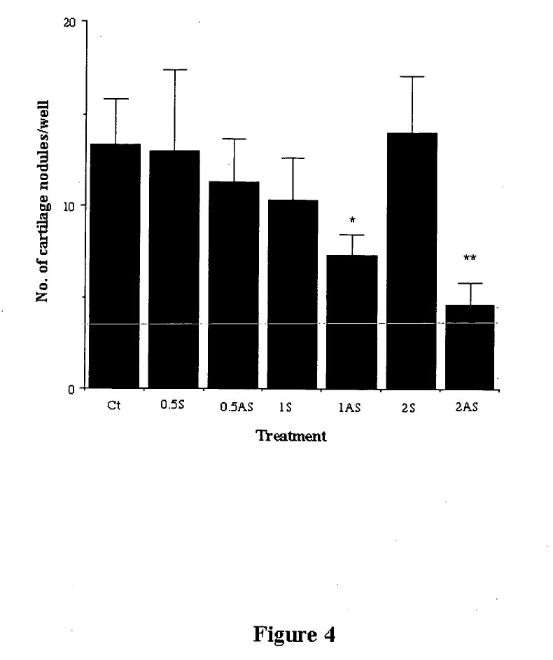

FIG. 4 illustrates a critical role for ERRα in cartilage formation, with down-regulation of cartilage nodule formation concomitant with down-regulation of ERRα expression in vitro. This result is independent of its effects on proliferation, since cartilage nodule formation was decreased when the antisense treatment commenced after proliferation had largely ceased. These results indicate an unexpected use for ERRα in the regulation of cartilage formation.

Another group of diseases involves unwanted or inappropriate cartilage formation. Such diseases include chondrosarcomas and chondrodysplasias. The present invention provides methods and pharmaceutical compositions for inhibiting cartilage formation by reducing ERRα activity and thereby treating such disorders. ERRα activity may be reduced by reducing the amount of ERRα protein being produced or by inhibiting the activity of ERRα protein. This may be achieved, for example, by administering an antisense sequence as described herein, or an agent which reduces ERRα expression, an antibody which binds specifically to ERRα protein or an ERRα antagonist. An ERRα antagonist is a compound which decreases the chondrogenic activity of ERRα protein.

An antisense sequence such as an antisense oligo or an antisense adenovirus can be administered by gene therapy as described above, preferably by local injection. Antibodies or antagonists can be administered locally, or systemically if target specific.

A number of ERRα antagonists have been described. For example, organochlorine compounds such as chlordane and toxaphene have been shown to antagonise ERRα activity (Yang et al., (1999)).

Diethylstilbestrol has also been described as an ERRα antagonist (Tremblay et al., (2001a)).

These compounds may be employed or may be used as a starting point for the development of analogues which can be screened as described herein for ERRα antagonist properties.

In a further embodiment, the invention provides a method for assessing the ERRα level or activity of a tissue, which can be used as a screening method for possible susceptibility to cartilage degeneration or as a method for monitoring treatment efficacy during treatment of a cartilage degenerative disorder. For example, subjects such as athletes or the overweight, who are at increased risk of osteo arthritis, could be screened for below normal cartilage ERRα , which would suggest susceptibility to development of osteo arthritis. Subjects being treated for rheumatoid arthritis could have their cartilage ERRα level monitored at intervals to assess whether normal ERRα levels were being restored or maintained. ERRα levels can be measured in samples of biopsied joint cartilage tissue, for example by RT-PCR of mRNA as described herein and in Bonnelye et al., (2001) or, less quantitatively, by immunolabelling techniques such as those described in Bonnelye et al., (2001).

The invention also provides a method for screening a candidate compound for its ability to modulate ERRα chondrogenic activity in a suitable system, by examining ERRα chondrogenic activity in the presence or absence of the candidate compound. A change in ERRα chondrogenic activity in the presence of the compound relative to ERRα chondrogenic activity in the absence of the compound indicates that the compound modulates ERRα chondrogenic activity. If ERRα chondrogenic activity is increased relative to the control in the presence of the compound, the compound is potentially useful as a stimulator of chondrogenesis. By means of the assays described herein, one of skill in the art can readily determine whether such a compound caused increased ERRα expression or acted as an ERRα agonist, to increase activity of ERRα protein. Conversely, if ERRα chondrogenic activity is decreased in the presence of the compound, relative to the control, the compound is potentially useful as an inhibitor of chondrogenesis. It can be determined by means of the assays described herein whether such a compound caused decreased ERRα expression or acted as an ERRα antagonist, to decrease activity of ERRα protein.

Any assay system which enables one to measure the chondrogenic activity or cartilage promoting activity of ERRα may be employed as the basis of the screening method. Suitable assay systems include, for example, measurement of chondroprogenitor proliferation, cartilage nodule formation or increase of chondroblast markers stimulated by increased ERRα expression in a chondrogenic cell line such as C5.18, as described herein.

Candidate compounds may be subjected to an initial screening for their effect on activation of the ERRα promoter, before proceeding to the more involved testing of their biological effect in the screening method described above. While ERRs do not respond to natural estrogens, these receptors recognise the estrogen response element and have been shown to activate and repress gene expression in the absence of endogenously added ligand. One of skill in the art can refer to Shi et al. (1997), Yang et al. (1999) and Tremblay et al. (2001) for suitable methods.

In accordance with a further embodiment of the invention, the ERRα signalling pathway may be modulated by modulating the binding of the ERRα to an ERRα binding partner. Such a binding partner may include for example the estrogen receptor. ERRα can be used to upregulate the transcription and thus expression of genes which work together with ERRα to affect cartilage development.

The invention further provides methods for screening candidate compounds to identify those able to modulate signaling by ERRα through a pathway involving ERRα.

For example, the invention provides screening methods for compounds able to bind to ERRα which are therefore candidates for modifying the chondrogenic activity of ERRα. Various suitable screening methods are known to those in the art, including immobilization of ERRα on a substrate and exposure of the bound ERRα to candidate compounds, followed by elution of compounds which have bound to the ERRα.

Co-immunoprecipitation of protein binding partners with an ERRα-specific antibody will allow the identification of cartilage-specific binding partners which contribute to ERRα chondrogenic activity.

The invention also provides a method of modulating a ERRα signaling pathway by increasing or decreasing the availability of ERRα or by modulating the function of the ERRα.

The invention further provides methods for preventing or treating diseases characterised by an abnormality in an ERRα signaling pathway which involves ERRα, by modulating signaling in the pathway.

According to another aspect of the present invention is a method for suppressing in a mammal, the proliferation of a chondrocytic cell capable of being stimulated to proliferate by ERRα , the method comprising administering to the mammal an effective amount of a ERRα antagonist or an antibody which binds specifically to ERRα.

The invention also enables transgenic non-human animal models, which may be used for study of the effects on chondrogenesis of over and under expression of the ERRα gene, for the screening of candidate compounds as potential agonists or antagonists of this receptor and for the evaluation of potential therapeutic interventions.

The transgenic animals of the invention may also provide models of disease conditions associated with abnormalities of ERRα expression. Animal species suitable for use in the animal models of the invention include mice, rats, rabbits, dogs, cats, goats, sheep, pigs and non-human primates.

Animal models may be produced which over-express ERRα by inserting a nucleic acid sequence encoding ERRα into a germ line cell or a stem cell under control of suitable promoters, using conventional techniques such as oocyte microinjection or transfection or microinjection into stem cells. A cartilage specific promoter such as the Type II collagen promoter may be used, for example. Animal models can also be produced by homologous recombination to create artificially mutant sequences (knock-in targeting of the ERRα gene) or loss of function mutations (knock-out targeting of the ERRα gene). For example, knock-out targeting of the ERRα gene). For example, knock-out animal models can be made using the tet-receptor system described U.S. Pat. No. 5,654,168 or the Cre-Lox system described, for example, in U.S. Pat. Nos. 4,959,717 and 5,801,030.

In accordance with one embodiment of the invention, transgenic animals are generated by the introduction of a ERRα transgene into a fertilized animal oocyte, with subsequent growth of the embryo to birth as a live animal. The ERRα transgene is a transcription unit which directs the expression of ERRα gene in eukaryotic cells. To create the transgene, ERRα gene is ligated with an eukaryotic expression module. The basic eukaryotic expression module contains a promoter element to mediate transcription of ERRα sequences and signals required for efficient for termination and polyadenylation of the transcript. Additional elements of the module may include enhancers which stimulate transcription of ERRα sequences. The most frequently utilized termination and polyadenylation signals are those derived from SV40. The choice of promoter and enhancer elements to be incorporated into the ERRα transgene is determined by the cell types in which ERRα gene is to be expressed. To achieve expression in a broad range of cells, promoter and enhancer elements derived from viruses may be utilized, such as the herpes simplex virus thymidine kinase promoter and polyoma enhancer. To achieve exclusive expression in a particular cell type, specific promoter and enhancer elements could be used, such as the promoter of the mb-1 gene and the intronic enhancer of the immunoglobulin heavy chain gene. In a preferred embodiment, a cartilage specific promoter such as the promoter of Type II collagen may be used to target expression in chondrocytes (Bridgewater 1998; Lefebvre 1996).

The ERRα transgene is inserted into a plasmid vector, such as pBR322 for amplification. The entire ERRα transgene is then released from the plasmid by enzyme digestion, purified and injected into an oocyte. The oocyte is subsequently implanted into a pseudopregnant female animal. Southern blot analysis or other approaches are used to determined the genotype of the founder animals and animals generated in the subsequent backcross and intercross.

Such deficient mice will provide a model for study of the role of ERRα in chondrocyte differentiation and proliferation and general skeletal development. Such animals will also provide tools for screening candidate compounds for their interaction with ERRα or the signalling pathway activated by ERRα.

The invention also provides pharmaceutical compositions for promoting cartilage formation, comprising as active ingredient a substantially purified ERRα protein, an ERRα agonist or an isolated nucleotide sequence encoding ERRα protein. Such compositions are useful, for example, in treating disorders associated with cartilage degeneration.

ERRα protein may be produced by conventional recombinant techniques permitting expression of ERRα by a suitable host cell. A DNA encoding ERRα may be prepared as described, for example, in Giguere et al. (1998).

Techniques for production of proteins by recombinant expression are well known to those in the art and are described, for example, in Sambrook et al. (1989) or latest edition thereof. Suitable host cells include E. coli or other bacterial cells, yeast, fungi, insect cells or mammalian cells.

The invention provides for compositions for promoting cartilage formation comprising as active ingredient an ERRα agonist obtained by using a screening method as described herein.

A nucleotide sequence encoding ERRα protein may be administered to a subject experiencing cartilage loss due to an absent or defective ERRα gene either in vivo or ex vivo. Expression may be targeted to a selected cell or tissue by use of an appropriate promoter.

The invention also provides pharmaceutical compositions for reducing or inhibiting cartilage formation, comprising as active ingredient an antibody which binds specifically to ERRα, an ERRα antagonist or a negative regulator such as an antisense nucleic acid or a dominant negative mutant version of the ERRα gene.

The invention provides for compositions for reducing cartilage formation comprising as active ingredient an ERRα antagonist obtained by using a screening method as described herein.

Antibodies which bind specifically to ERRα protein may be made by conventional techniques.

The term “antibodies” includes polyclonal antibodies, monoclonal antibodies, single chain antibodies and fragments such as Fab fragments.

In order to prepare polyclonal antibodies, fusion proteins containing defined portions or all of an ERRα protein can be synthesized in bacteria by expression of the corresponding DNA sequences, as described above. Fusion proteins are commonly used as a source of antigen for producing antibodies. Alternatively, the protein may be isolated and purified from the recombinant expression culture and used as source of antigen. Either the entire protein or fragments thereof can be used as a source of antigen to produce antibodies.

The purified protein is mixed with Freund's adjuvant and injected into rabbits or other appropriate laboratory animals. Following booster injections at weekly intervals, the animals are then bled and the serum isolated. The serum may be used directly or purified by various methods including affinity chromatography to give polyclonal antibodies.

Monoclonal anti-ERRα antibodies may be produced by methods well known in the art. Briefly, the purified protein or fragment thereof is injected in Freund's adjuvant into mice over a suitable period of time, spleen cells are harvested and these are fused with a permanently growing myeloma partner and the resultant hybridomas are screened to identify cells producing the desired antibody. Suitable methods for antibody preparation may be found in standard texts such as Barreback, E. D. (1995).

The pharmaceutical compositions of the invention may comprise, in addition to the active ingredient, one or more pharmaceutically acceptable carriers.

Administration of an effective amount of a pharmaceutical composition of the present invention means an amount effective, at dosages and for periods of time necessary to achieve the desired result. This may also vary according to factors such as the disease state, age, sex, and weight of the subject, and the ability of the composition to elicit a desired response in the subject. Dosage regima may be adjusted to provide the optimum therapeutic response. For example, several divided doses may be administered daily or the dose may be proportionally reduced as indicated by the exigencies of the therapeutic situation.

By pharmaceutically acceptable carrier as used herein is meant one or more compatible solid or liquid delivery systems. Some examples of pharmaceutically acceptable carriers are sugars, starches, cellulose and its derivatives, powdered tragacanth, malt, gelatin, collagen, talc, stearic acids, magnesium stearate, calcium sulfate, vegetable oils, polyols, agar, alginic acids, pyrogen-free water, isotonic saline, phosphate buffer, and other suitable non-toxic substances used in pharmaceutical formulations. Other excipients such as wetting agents and lubricants, tableting agents, stabilizers, anti-oxidants and preservatives are also contemplated.

The compositions described herein can be prepared by known methods for the preparation of pharmaceutically acceptable compositions which can be administered to subjects, such that an effective quantity of the active substance is combined in a mixture with a pharmaceutically acceptable carrier. Suitable carriers and formulations adapted for particular modes of administration are described, for example, in Remington's Pharmaceutical Sciences (Remington's Pharmaceutical Sciences, Mack Publishing Company, Easton, Pa., USA 1985). On this basis the compositions include, albeit not exclusively, solutions of the substance in association with one or more pharmaceutically acceptable vehicles or diluents, and contained in buffered solutions with a suitable pH and iso-osmotic with the physiological fluids.

The pharmaceutical compositions of the invention may be administered therapeutically by various routes such as by injection or by oral, nasal, intra-articular, intra-vertebral, buccal, rectal, vaginal, transdermal or ocular administration in a variety of formulations, as is known to those skilled in the art.

The present invention enables also a screening method for compounds of therapeutic utility as antagonists of the chondrogenic activity of ERRα. Such antagonist compounds are useful, for example, to reduce or prevent differentiation and maturation of chondrocytes. ERRα antagonists may also be used in the treatment of cartilage related disorders involving inappropriate cartilage growth. Those skilled in the art will be able to devise a number of possible screening methods for screening candidate compounds for ERRα antagonism.

A screening method may also be based on binding to the ERRα receptor. Such competitive binding assays are well known to those skilled in the art. Once binding has been established for a particular compound, a biological activity assay is employed to determine agonist or antagonist potential.

### EXAMPLES

The examples are described for the purposes of illustration and are not intended to limit the scope of the invention.

Methods of biochemistry, molecular biology, histology and immunology referred to but not explicitly described in this disclosure and examples are reported in the scientific literature and are well known to those skilled in the art.

### Example 1

### Expression of ERRα in Chondrocyte Lineage Cells Throughout Development

In order to assess ERRα expression, RCJ.1C5.18 (C5.18) cells were grown as described by Grigoriadis, 1996. This cell line is a fetal rat cell line which undergoes differentiation into cartilage-producing chondrocytes; it is widely used as a model system for the study of chondrogenesis and the regulation of chondrocyte activity. Cells were maintained in α-MEM containing 15% heat-inactivated FBS (Flow Laboratories, McLean, Va.), antibiotics comprising 100 μg/ml penicillin G (Sigma Chemical Co., St. Louis, Mo.), 50 μg/ml gentamycin (Sigma), and 0.3μg/ml fungizone (Flow Laboratories) and 10⁻⁸M dexamethasone (Merck, Sharp, and Dohme, Canada, Ltd., Kirkland, PQ). Dexamethasone (Dex) stimulates chondrogenesis and cartilage formation in these cultures. For differentiation studies, cells were grown in the same medium, with or without dexamethasone, and with the addition of 50 μg/ml ascorbic acid and 10 mM sodium β-glycerophosphate. Medium was changed every 2 days. All dishes were incubated at 37° C. in a humidified atmosphere in a 95% air/5% CO₂ incubator.

For Northern blot analysis, total RNA was extracted with guanidine from C5.18 cells after culture periods corresponding to different stages of proliferation, differentiation and cartilage nodule formation. Northern blots were prepared and hybridized with a 750 bp fragment corresponding to the rat 3′ UTR of ERRα (provided by J M Vanacker, Lyon, France) according to standard procedures (Chirgwin et al, 1979). Mouse cDNAs for link protein and aggrecan were kindly provided by S. Bernier, London, On.

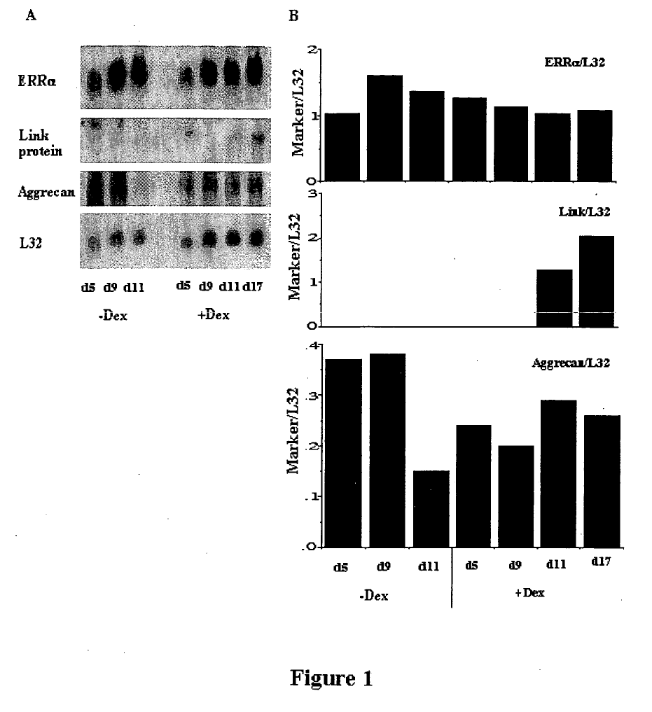

ERRα mRNA expression levels were assessed over a proliferation-differentiation time course by Northern blotting of C5.18 cells grown in the presence (+Dex) or absence (−Dex) of dexamethasone), a stimulator of differentiation in this model. Under both growth conditions, ERRα mRNA was expressed constitutively at all times assessed, including proliferation (day 5), and early (day 9, 11) and late (day 17) cartilage nodule formation, as shown in Panel A of FIG. 1. For comparison, levels of mRNA levels for two cartilage markers, aggrecan and link protein are shown in Panel B of FIG. 1.

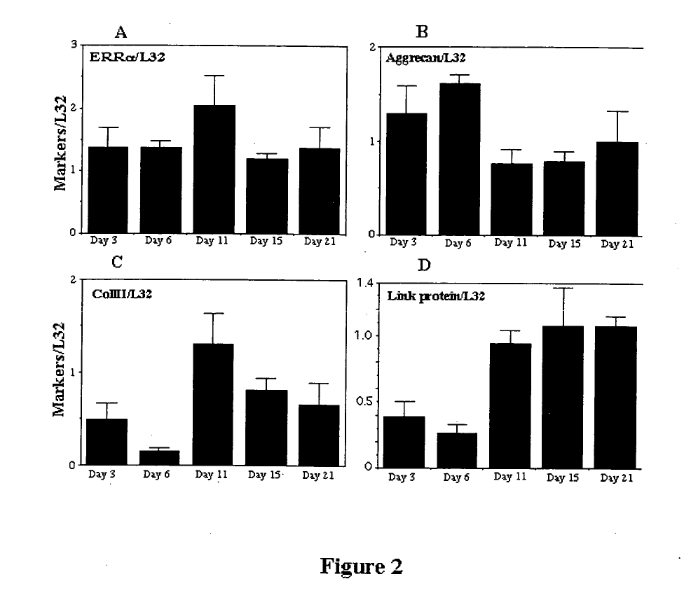

Semi-quantitative RT-PCR was carried out as described in Bonnelye et al. (2001), over the proliferation/differentiation time course of C5.18 cultures treated with Dex. The results are shown in FIG. 2. Days 3 and 6 were within the proliferation phase and days 11, 15 and 21 were within the differentiation stage. ERRα expression was normalised against that of the ribosomal probe L32. For comparison, mRNA levels for three chondroblast markers, type II collagen, aggrecan and link protein, were also assessed, normalised against L32.

In order to assess ERRα protein expression, C5.18 cell cultures, grown as described above, were immunolabelled essentially as described previously [Turksen, 1991; Turksen, 1992]. Cultures were rinsed with PBS, fixed with 3.7% formaldehyde in PBS and permeabilized with methanol at −20° C. Frozen sections were fixed 10 min in cold acetone. Paraffin sections were deparaffinized in xylene, then rehydrated in 100%, 95% and 70% ethanol and water. After rinsing, cells in dishes or frozen or paraffin sections were incubated for 1 h at room temperature with 10% normal serum in PBS for ERRα and ERα and in 3% BSA in PBS (denaturated) for ERα. After rinsing, cells or sections were incubated for 1.5 hours with appropriate dilutions of primary antibodies (1/50, anti-ERRα; anti-ERα or anti- ERβ MC-20 or Y-19, respectively; Santa Cruz Biotechnology, Inc).

While ERRα protein was expressed throughout the proliferation/differentiation sequence, protein levels were highest in maturing and mature chondrocytes associated with cartilage nodules in vitro. At all stages (proliferation and differentiation/maturation), the majority of ERRα appeared to be localized to the nucleus. For comparison, ERα and ERβ levels were also assessed; all three receptors were co-expressed in at least some chondrocytes. However, based on staining intensity, ERRα levels were highest (detected in all chondrocytes), followed by ERα (detectable in most chondrocytes but at lower levels than ERRα ) and finally by ERβ (detectable at very low levels in only a subset of chondrocytes). As with ERRα, ERα appeared to be primarily localized to the nucleus at all stages, whereas ERβ appeared to assume a nuclear localization mainly when cells were in proliferative stages (data not shown).

### Example 2

### In Vivo Expression of ERRα

To determine the in vivo expression of ERRα protein, immuno-cytochemistry was performed on sections of 21 day fetal rat tibiae and metatarsals and on sections of adult rat tibiae and femurs. The sections were rinsed in PBS and incubated for 1 h at room temperature with secondary antibody CY-3-conjugated anti-rabbit (Jackson immunoresearch Lab, West Grove, Pa., USA; 1/300 final dilution) for ERRα . After rinsing, samples were mounted in Moviol (Hoechst Ltd, Montreal, PQ, Canada) and observed by epifluorescence microscopy on a Zeiss Photomicroscope III (Zeiss, Oberkochen, Germany).

ERRα protein was already highly expressed in the chondrocytes of the growth plates of term-pregnant rat fetuses and continued to be expressed in the cartilage of adult animals. In fetal growth plate cartilage, intense label for ERRα was seen in perichondrial precursors and proliferating chondrocytes, while staining in hypertrophic chondrocytes was low or absent. In adult animals, growth plate chondrocytes, including hypertrophic zone, were intensely labeled, as were articular chondrocytes. Articular zone chondrocytes, based on labeling intensity, expressed much higher levels of ERRα than cells in surrounding tissues (data not shown).

### Example 3

### Antisense and Sense Oligonucleotide Treatment

Antisense oligonucleotides form DNA:RNA duplexes with specific mRNA species, thereby blocking binding of the mRNA to the 40S ribosomal subunit and preventing translation [Jen, 2000]. To examine the involvement of ERRα in chondrocyte differentiation and cartilage formation, C5.18 cells were treated either during the proliferation phase or during the differentiation and cartilage nodule formation phase. Preliminary experiments were done to determine effective oligonucleotide concentrations that were not toxic (not shown) and the efficacy of the antisense was confirmed by immunocytochemistry and Western blots.

C5.18 cells were plated in 24 wells plates at 10⁴ cells/well and treated with antisense or sense oligonucleotides. Antisense oligonucleotide inhibition of ERRα expression was accomplished with a 20-base phosphorothioate-modified oligonucleotide, localized to the A/B domain. The ERRα antisense oligonucleotide sequence was: 5′-TCACCGGGGGTTCAGTCTCA-3′. Control dishes were treated with the complementary sense oligonucleotide or no oligonucleotide. Preliminary experiments were done to determine effective oligonucleotide concentrations that were not toxic. 0.1 μM to 5 μM oligonucleotides were added directly to cells either during the proliferation phase (days 1 to 4) and 0.5 μM to 2 μM oligonucleotides were added during the differentiation phase (day 5 to day 13) in standard medium as above supplemented with 50 μg/ml ascorbic acid, 10 mM sodium β-glycerophosphate, and 10⁻⁸ M dexamethadone. Medium was changed every 2 days and fresh oligonucleotides were added.

For cell growth analysis, the cell layers were rinsed in PBS, released with trypsin and collagenase, and the harvested cells were counted electronically. The results are shown in FIG. 3. Results are plotted as the average of three counts for each of three wells for control and each concentration of antisense or sense primers used. When C5.18 cells were treated between days 1-4, the proliferation stage, a significant and specific dose-dependent decrease in cell number in dishes treated with antisense compared to sense or untreated controls is seen. These results indicate a role for ERRα in the proliferation or very early differentiation phases of C5.18 cells.

To determine whether ERRα also plays a role in chondrocyte differentiation independently of an effect on proliferation, C5.18 cells were treated with the antisense oligonucleotide beginning at day 5 (after cells had reached confluence and proliferation was decreased) to day 15. For quantification of cartilage formation, dishes or wells were fixed and with Alcian blue and cartilage nodules were counted on a grid [Grigoriadis, 1996]. Results, as shown in FIG. 4, are plotted as the mean number of nodules ±SD of three wells for controls and each concentration of antisense or sense primers. A striking dose-dependent decrease in cartilage nodule formation was seen.

### Example 4

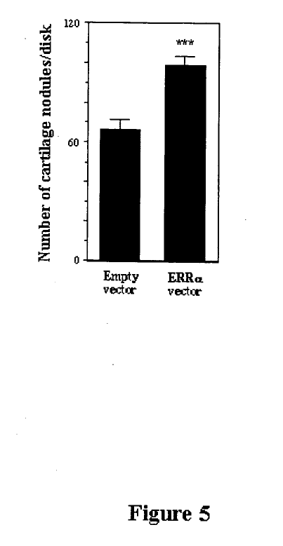

C5.18 cells at ˜50% confluency were transfected with either a pcDNA3 empty plasmid (Empty vector) or pcDNA3-ERRα (ERRα vector) (0.5 μg DNA per transfection). Five 35-mm dishes per treatment group were fixed, stained with Alcian blue and the cartilage nodules counted. Cultures transfected with the ERRα gene showed a significant increase in the number of cartilage nodules formed (mean +/−SD; Welch test or Mann Whitney test, p<0.05). Results shown in FIG. 5 are representative of two independent experiments.

### Example 5

### Rat Model of Inflammatory Arthritis

Arthritis was initiated in genetically susceptible female Lewis rats (Charles River Breeding Laboratories, Wilmington, Mass.) by intraperitoneal injection of group A SCW (Streptococcal Cell Wall) peptidoglycan-polysaccharide complexes (Lee Laboratories Inc., Grayson, Ga.) as described [Wahl, 1994]. The severity of arthritis (articular index, AI) was determined by blinded scoring of each ankle and wrist joint based on the degree of swelling, erythema, and distortion on a scale of 0-4 and summing the scores for all four limbs. As a control, PBS was injected instead of SWC. Hemoglobin as an NO scavenger, was used as a suppressor of arthritis. In one group of rats, it was administered daily from day 0 to day 24 of SCW treatment and in a second group from day 10 to day 24 only.

ERRα expression in chondrocytes was examined in sections of articular cartilage, compact bone and bone marrow from control rats and from SCW induced arthritic rats by immunocytochemistry as described above.

In the Streptococcal cell wall (SCW)-induced rat arthritis model, ERRα expression was decreased in chondrocytes in the eroding articular cartilage as a function of the severity of the disease.

Semi-quantitative RT-PCR as described in example 1 was used to determine mRNA levels in samples from a further rat model and a mouse model of collagen-induced arthritis (Trentham et al. (1997)), to assess ERRα expression.

RNA was isolated with Trizol reagent (Gibco BRL), according to the manufacturer's protocol, from joints from three control rats and from three rats injected with collagen; joints comprised proximal femur and- distal tibiae. Semi-quantitative RT-PCR with ERRα -specific primers was done and ERRα mRNA expression level was normalized against that of the ribosomal housekeeping gene L32.

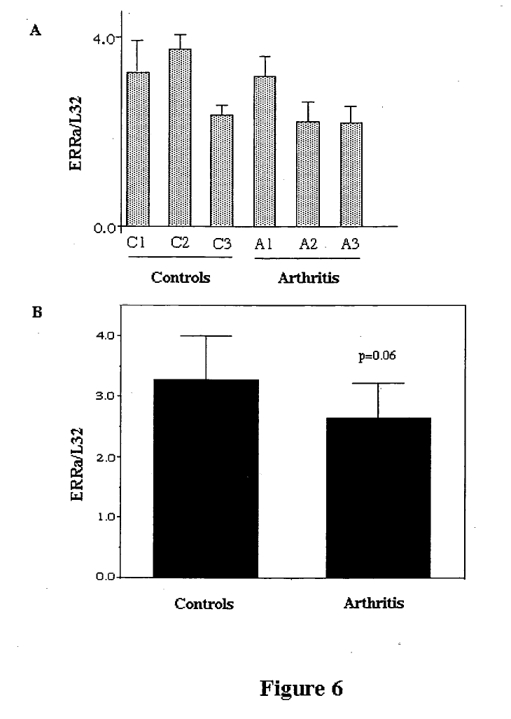

In the rat model, ERRα mRNA levels were reduced in arthritic joints compared to normal joints, as seen in FIG. 6 (your FIG. 9).

Samples obtained from the mouse model consisted of separated bone and joint samples from 3 arthritic mice and 3 controls. Joints (proximal femur and distal tibiae) were carefully dissected away from bone and ERRα expression was separately determined for cartilage tissue and for bone (left and right tibiae and femurs, marrow removed). The six normal cartilage samples were pooled for analysis, as were the six arthritic cartilage samples.

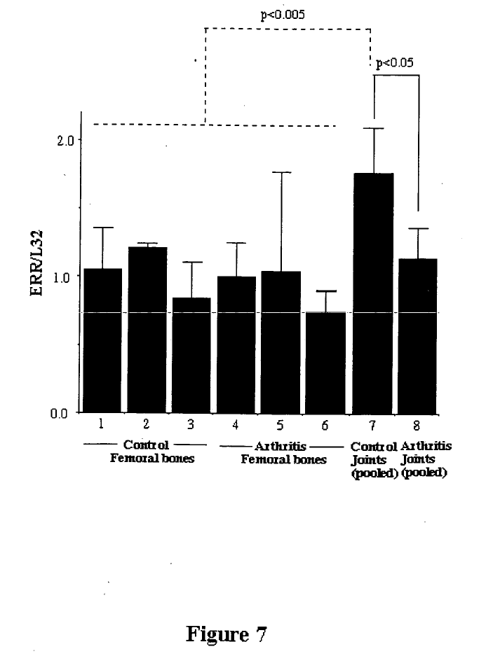

RNA was isolated from the tissues, semi-quantitative RT-PCR with ERRα -specific primers was done and ERRα mRNA expression level was normalized against that of the ribosomal housekeeping gene L32. ERRα mRNA levels were significantly reduced in pooled arthritic joint cartilage samples compared with those from control samples (Student t-test; p<0.05) as seen in FIG. 7 (your FIG. 10). ERRα mRNA levels were higher in control joint cartilage than in control bone (Student t-test; p<0.005). In contrast, no significant difference in ERRα expression level was seen between control and arthritic bone samples.

### Example 6

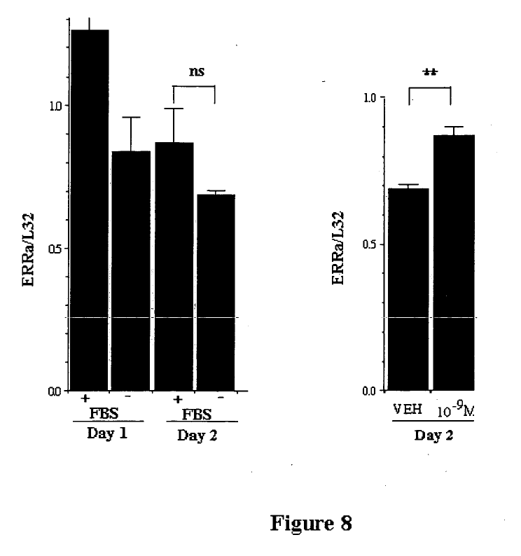

C5.18 cells were grown in medium with (+) and without (−) fetal bovine serum (FBS). ERRα mRNA levels were determined by semi-quantitative RT-PCR and normalised against ribosomal probe L32. As shown in FIG. 8, Panel A, ERRα mRNA was increased by the presence of FBS at days 1 and 2.

Similar C5.18 cultures were grown without FBS but in the presence of estrogen (10⁻⁹M E2) or vehicle (0.01% ethanol) (VEH) and mRNA levels were determined as above. As shown in FIG. 8, Panel B, estrogen significantly increased ERRα mRNA levels at day 2. Estrogen plus FBS did not increase the ERRα mRNA level over that seen with FBS alone, suggesting that the latter caused maximal stimulation of ERRα expression.

The present invention is not limited to the features of the embodiments described herein, but includes all variations and modifications within the scope of the claims.

### REFERENCES

Bonnelye, E., Vanacker, J. M., Dittmar, T., Begue, A., Desbiens, X., Denhardt, D. T., Aubin, J. E., Laudet, V., and Fournier, B. (1997a). The ERR-1 orphan receptor is a transcriptional activator expressed during bone development. Mol Endocrinol 11(7), 905-16.
Bonnelye, E., Vanacker, J. M., Spruyt, N., Alric, S., Fournier, B., Desbiens, X., and Laudet, V. (1997b). Expression of the estrogen-related receptor 1 (ERR-1) orphan receptor during mouse development. Mech Dev 65(1-2), 71-85.
Bonnelye et al., (2001), J. Cell Biol., v. 153, pp. 971-983.
Bridgewater, L. C., Lefebvre, V., and de Crombrugghe, B. (1998) Chodrocyte-specific enhancer elements in the Col11a2 gene resemble the Col2a1 tissue-specific enhancer. J Biol Chem 273(24), 14998-15006.
Denhardt, D. T., and Noda, M. (1998). Osteopontin expression and function: role in bone remodeling. 25th Anniversary Issue. New directions and dimensions in cellular biochemistry. J. Cell. Biochem. Suppl. 30/31, 92-102.
Enmark, E., and Gustafsson, J. A. (1996). Orphan nuclear receptors—the first eight years. Mol Endocrinol 10(11), 1293-307.
Gelse et al., (2001), Arthritis Rheum., v. 44, pp. 1943-53.
Giguere, V., Yang, N., Segui, P., and Evans, R. M. (1988). Identification of a new class of steroid hormone receptors. Nature 331(6151), 91-4.
Goater et al., (2000), J. Rheumatol., v. 27, pp. 983-989.
Green, S., Walter, P., Kumar, V., Krust, A., Bornert, J. M., Argos, P., and Chambon, P. (1986). Human oestrogen receptor cDNA: sequence, expression and homology to v-erb-A. Nature 320(6058), 134-9.
Grigoriadis, A. E., Heersche, J. N., and Aubin, J. E. (1996). Analysis of chondroprogenitor frequency and cartilage differentiation in a novel family of clonal chondrogenic rat cell lines. Differentiation 60(5), 299-307.
Gronemeyer, H., and Laudet, V. (1995). Transcription factors 3: nuclear receptors. Protein Profile 2(11), 1173-308.
Hong, H., Yang, L., and Stallcup, M. R. (1999). Hormone-independent transcriptional activation and coactivator binding by novel orphan nuclear receptor ERR3. J Biol Chem 274(32), 22618-26.
Jen, K. Y., and Gewirtz, A. M. (2000). Suppression of gene expression by targeted disruption of messenger RNA: available options and current strategies. Stem Cells 18(5), 307-319.
John, S., Myerscough, A., Eyre, S., Roby, P., Hajeer, A., Silman, A. J., Ollier, W. E., and Worthington, J. (1999). Linkage of a marker in intron D of the estrogen synthase locus to rheumatoid arthritis. Arthritis Rheum 42(8), 1617-1620.
Kuiper, G. G., Enmark, E., Pelto-Huikko, M., Nilsson, S., and Gustafsson, J. A. (1996). Cloning of a novel receptor expressed in rat prostate and ovary. Proc Nat Acad Sci USA 93(12), 5925-30.
Lefebvre, V., Zhou, G., Mukhopadhyay, K., Smith, C. N., Zhang, Z., Eberspaecher, H., Zhou, X., Sinha, S., Maity, S. N., and de Crombrugghe, B. (1996). An 18-base-pair sequence in the mouse proalphal 1(II) collagen gene is sufficient for expression in cartilage and binds nuclear proteins that are selectively expressed in chondrocytes. Mol Cell Biol 16(8), 4512-4523.
McDougall, S., Fu, Y. H., Lowe, G. N., Williams, A., Polendo, R., Benya, P. D., lida-Klein, A., Fang, M. A., and Hahn, T. J. (1996). Surface adhesion-mediated regulation of chondrocyte-specific gene expression in the nontransformed RCJ 3.1C5.18 rat chondrocyte cell line. J Bone Miner Res 11(8), 1130-1138.
Nixon et al., (2000), Clin. Orthop., 379 Suppl, S 201-13.
Pettersson, K., Svensson, K., Mattsson, R., Carlsson, B., Ohlsson, R., and Berkenstam, A. (1996). Expression of a novel member of estrogen response element-binding nuclear receptors is restricted to the early stages of chorion formation during mouse embryogenesis. Mech Dev 54(2), 211-23.
Sasano, H., Uzuki, M., Sawai, T., Nagura, H., Matsunaga, G., Kashimoto, O., and Harada, N. (1997). Aromatase in human bone tissue. J Bone Miner Res 12(9), 1416-1423.
Shi et al., (1997), Genomics, v. 44, pp. 52-60
Simpson, E., Rubin, G., Clyne, C., Robertson, K., O'Donnell, L., Jones, M., and Davis, S. (2000). The Role of Local Estrogen Biosynthesis in Males and Females. Trends Endocrinol Metab 11 (5), 184-188.
Simpson, E. R., Zhao, Y., Agarwal, V. R., Michael, M. D., Bulun, S. E., Hinshelwood, M. M., Graham-Lorence, S., Sun, T., Fisher, C. R., Qin, K., and Mendelson, C. R. (1997). Aromatase expression in health and disease. Recent Prog Horm Res 52, 185-213; discusslion 213-214.
Tremblay et al., (2001a), Genes Dev., v. 15, pp. 833-838.
Tremblay et al., (2001b), Endocrinol., v. 142, pp. 4572-5.
Trentham et al., (1977), J. Exp. Med., v. 146, pp. 857-868.
Turksen, K., and Aubin, J. E. (1991). Positive and negative immunoselection for enrichment of two classes of osteoprogenitor cells. J. Cell Biol. 114(2), 373-384.
Turksen, K., Bhargava, U., Moe, H. K., and Aubin, J. E. (1992). Isolation of monoclonal antibodies recognizing rat bone-associated molecules in vitro and in vivo. J. Histochem. Cytochem. 40(9), 1339-1352.
Vanacker, J. M., Delmarre, C., Guo, X., and Laudet, V. (1998). Activation of the osteopontin promoter by the orphan nuclear receptor estrogen receptor related alpha. Cell Growth Differ 9(12), 1007-1014.
Vanderschueren, D., Boonen, S., Ederveen, A. G., de Coster, R., Van Herck, E., Moermans, K., Vandenput, L., Verstuyf, A., and Bouillon, R. (2000). Skeletal effects of estrogen deficiency as induced by an aromatase inhibitor in an aged male rat model [In Process Citation]. Bone 27(5), 611-617.
Vanderschueren, D., Van Herck, E., De Coster, R., and Bouillon, R. (1996). Aromatization of androgens is important for skeletal maintenance of aged male rats. Calcif Tissue Int 59(3), 179-183.
van Lent et al., (2002), Osteoarthritis Cartilage, v. 10, pp. 234-243.
Wahl, S. M., Allen, J. B., Hines, K. L., Imamichi, T., Wahl, A. M., Furcht, L. T., and McCarthy, J. B. (1994). Synthetic fibronectin peptides suppress arthritis in rats by interrupting leukocyte adhesion and recruitment. J Clin Invest 94(2), 655-662.
Yang, C., and Chen, S. (1999). Two organochlorine pesticides, toxaphene and chlordane, are antagonists for estrogen-related receptor alpha-1 orphan receptor. Cancer Res 59(18), 4519-24.
Yang, C., Zhou, D., and Chen, S. (1998). Modulation of aromatase expression in the breast tissue by ERR alpha-1 orphan receptor. Cancer Res 58(24), 5695-700.
Yang, N., Shigeta, H., Shi, H., and Teng, C. T. (1996). Estrogen-related receptor, hERR1, modulates estrogen receptor-mediated response of human lactoferrin gene promoter. J Biol Chem 271(10), 5795-804.
Zhang, Z., and Teng, C. T. (2000). Estrogen receptor-related receptor alpha 1 interacts with coactivator and constitutively activates the estrogen response elements of the human lactoferrin gene. J Biol Chem 275(27), 20837-46.

We claim:

1. A method of promoting cartilage formation in a mammal comprising administering to the mammal an effective amount of an agent selected from the group consisting of:
(a) an estrogen receptor related receptor alpha (ERRα) agonist;
(b) a substantially purified ERRα protein
(c) a nucleotide sequence encoding ERRα protein; and
(d) an agent which enhances expression of a gene encoding an ERRα protein.

2. The method of claim 1 wherein the agent increases proliferation of chondroprogenitor cells and chondrocytes.

3. The method of claim 1 wherein the agent promotes differentiation of chondroprogenitor cells and chondroblasts.

4. The method of claim 1 wherein the mammal suffers from a condition selected from the group consisting of cartilage loss, cartilage degeneration and cartilage injury.

5. The method of claim 1 wherein the mammal suffers from arthritis.

6. The method of claim 1 wherein the mammal suffers from a disease selected from the group consisting of ankylosing spondylitis, childhood arthritis, chronic back injury, gout, infectious arthritis, osteoarthritis, osteoporosis, pagets's disease, polymyalgia rheumatica, pseudogout, psoriatic arthritis, reactive arthritis, reiter's syndrome, repetitive stress injury, and rheumatoid arthritis.

7. The method of claim 1 wherein the agent is administered systemically or orally.

8. The method of claim 1 wherein the agent is administered intra-articularly.

9. The method of claim 1 wherein the agent is estrogen.

10. A method of inhibiting cartilage formation in a mammal comprising administering to the mammal an effective amount of an agent selected from the group consisting of:
(a) an ERRα antagonist;
(b) a purified antibody which binds specifically to an ERRα protein;
(c) an antisense nucleotide sequence complementary to and capable of hybridizing to a nucleotide sequence encoding an ERRα protein; and
(d) an agent which reduces expression of a gene encoding an ERRα protein.

11. The method of claim 10 wherein the agent reduces proliferation of chondroprogenitor cells and chondrocytes.

12. The method of claim 10 wherein the agent reduces differentiation of chondroprogenitor cells and chondroblasts.

13. The method of claim 10 wherein the mammal suffers from chondrosarcoma or chondrodysplasia.

14. The method of claim 10 wherein the agent is administered systemically or orally.

15. The method of claim 10 wherein the agent is administered intra-articularly.

16. A method for promoting cartilage formation in a tissue or cell in vitro comprising contacting the tissue or cell with an agent selected from the group consisting of:
(a) an ERRα agonist;
(b) a substantially purified ERRα protein;
(c) a nucleotide sequence encoding ERRα protein or an effective portion thereof; and
(d) an agent which enhances expression of a gene encoding an ERRα protein.

17. The method of claim 16 wherein the tissue is a cartilage biopsy.

18. The method of claim 16 wherein the agent is estrogen.

19. A method for screening a candidate compound for its ability to modulate ERRα cartilage promoting activity comprising:
(a) providing an assay system for measuring cartilage formation; and
(b) measuring the cartilage promoting activity of ERRα in the presence or absence of the candidate compound,
wherein a change in ERRα cartilage promoting activity in the presence of the compound relative to ERRα cartilage promoting activity in the absence of the compound indicates an ability to modulate ERRα cartilage promoting activity.

20. The method of claim 19 wherein the change in ERRα cartilage promoting activity in the presence of the compound is an increase in ERRα cartilage promoting activity.

21. The method of claim 19 wherein the change in ERRα cartilage promoting activity in the presence of the compound is a decrease in ERRα cartilage promoting activity.

22. Use of a compound identified by the method of claim 20 for preparation of a medicament for promoting cartilage formation in a mammal.

23. Use of a compound identified by the method of claim 21 for preparation of a medicament for inhibiting cartilage formation in a mammal.


SEQUENCE LISTING (1 sequence)

SEQ ID NO: 1 - DNA, 20 bp, Artificial
  Antisense oligonucleotide
tcaccgggggttcagtctca